(19) United States
(12) Patent Application Publication
(10) Pub. No.: US 2005/0060194 A1
(43) Pub. Date: Mar. 17, 2005

(54) Method and system for monitoring health of an individual

(76) Inventor: Stephen J. Brown, Woodside, CA (US)

Correspondence Address: Lewis Morgan, 2 Palo Alto Square, 3000 El Camino Real, Suite 700, Palo Alto, CA, 94306, US

(21) Appl. No.: 10/818,879

(22) Filed: Apr. 5, 2004

Related U.S. Application Data
(60) Provisional application No. 60/460,708, filed on Apr. 4, 2003.

(51) Int. Cl. G06F 17/60; A61B 5/00

(52) U.S. Cl. 705/2

(57) ABSTRACT

Embodiments of the present invention include a system for improving the general personal health of individuals by networking a plurality of individuals who with remote systems, preferably personal computers that are in communication with a central server through the internet. The remote systems also communicate with external servers storing documents relating to health information, non-health information, and entertainment content. In one embodiment of the present invention collect data relating to an individual's demography, personal health, familial health and personalization factors. Using this information, the system generates a health and preferred news and entertainment content profile of the individual. Embodiments thereby provide a healthcare information portal that provides directed healthcare information and other information to attract the individual's attention.

### CLAIM OF PRIORITY This application claims the priority of U.S. Provisional Application No. 60/460,708 filed Apr. 4, 2003 which is incorporated herein by reference.

### FIELD OF THE INVENTION

This invention relates to healthcare and, in particular, to an improved method and system of monitoring a patient's condition.

### BACKGROUND OF THE INVENTION

Lifestyle factors contribute to the development of many chronic diseases, and their associated morbidity and mortality. Many of these chronic health conditions may be prevented, and an individual's span and quality of life improved through changes in lifestyle, such as consuming a healthy diet, exercising regularly and eliminating the abuse of tobacco and alcohol.

In particular, cigarette smoking is the single most preventable cause of premature death in the United States. More than 430,000 Americans die each year from smoking-related illness, translating to one in every five deaths. An additional 110,000 people die of causes related to alcohol abuse.

Obesity is another major cause of morbidity and mortality in the United States. More than half of all adults in the United States are considered overweight or obese. Far from being a purely cosmetic disorder, obesity substantially increases morbidity and impairs the quality of life in affected individuals. It is also a risk factor for chronic diseases including hypertension, coronary heart disease, Type II diabetes, gallbladder disease, osteoarthritis and cancers of the breast, colon, and uterus. Thus it is important to reduce body weight among obese and overweight individuals, and to prevent further weight gain in both normal and overweight individuals.

Physical inactivity, defined as the absence of leisure time physical activity such as recreational exercise, is another major lifestyle-related risk factor for chronic health related conditions. In the United States and other developed countries, industrial automation has caused the majority of the population to be involved in sedentary occupations. Chronic conditions related to inactivity include coronary heart disease, hypertension, Type II diabetes, depression anxiety, osteoporotic hip fractures and obesity.

Lifestyle modifications alone successfully can be used to treat many chronic health conditions, without resorting to the usage of medication. For instance, mild hypertension is often controlled through lifestyle changes such as dietary modification, weight reduction, stress control, and physical activity. Weight control in itself delays and even prevents the onset of Type II Diabetes and mild hypertension.

In addition to lifestyle factors, the risk of developing many chronic health conditions is affected by a family history of disease. Diseases such as cancer, heart disease, elevated lipids, obesity, diabetes, stroke, high blood pressure, alcoholism, mental illness, and allergies all are affected by heredity. It is not possible for an individual to change his or her genetic constitution; however, an individual knowing that a close relative has been afflicted by a certain disease can alert the individual and/or his or her healthcare provider to a potential problem. It is important for the provider to know that the individual's close relation was afflicted with a certain disease, as this individual may need more frequent tests or some form of preventive treatment. In addition, screening tests may be performed to detect the condition at an earlier and potentially more treatable stage. For instance, although screening mammograms are not recommended in the general population until age 50, screening mammograms are recommended from age 40 for a woman whose mother or sister developed breast cancer. Further, in such individuals, simple precautions relating to lifestyle factors can serve to decrease the risk of developing the chronic disease condition.

Although many people believe that healthy individuals tend to be healthy in every aspect of his or her life, individuals who maintain healthy behavioral patterns and habits in one attribute are not necessarily free from risky behavioral patterns in other attributes.

The health care community has recognized in recent years the importance of preventive care in managing the health of individuals. By year 2025, one-fourth of all Americans will be 60 years of age or older, and the modification of lifestyles and health related behaviors is likely to assume an increasingly greater significance with the passage of time. Preventive care is important for managing the health of patients having chronic diseases or long-term conditions, as well as for reducing the incidence of smoking and other undesirable behaviors in at-risk individuals.

Successful preventive care includes educating individuals about diseases and other consequences of unhealthy behavior. A vital part of preventative care, therefore, is improving, maintaining, and ensuring clear communication between individuals and caregivers, and providing individuals with treatments and other tools for managing diseases or behaviors.

Unfortunately, traditionally-used preventive care and health education approaches suffer from several drawbacks. The mass-marketing techniques used for health education by most health maintenance organizations (HMOs) and insurance companies allow for little customization of information to an individual's needs. Consequently, many individuals do not directly identify with the educational approaches used by their healthcare providers, reducing their effectiveness. Personalizing health education significantly raises the effectiveness of preventive health care, especially in children and adolescents. This is particularly relevant since most lifestyle behaviors and habits of an individual are acquired at this age.

Similarly, although there is a vast amount of health education material in print, in the electronic media, and on the Internet, this content is neither customized to the individual's health and personal profile nor is it presented to the individual in a way that prioritizes the information with regard to its relevance to the individual. For an example, a general search on the Internet may return information about a healthcare study, but that study may not be relevant to the individual demographically. Moreover, creators of websites and the health section editors of general magazines often paraphrase abstracts of articles in medical journals and change technical terminology to language understood by the laity. As a result, not only will an individual potentially stumble into masses of irrelevant information, but the irrelevance of it may be obscured by those trying to disseminate healthcare information to a mass audience.

As a result, the unavailability of relevant, directed information reduces the likelihood of an individual benefiting from the available educational material, or incorporating such knowledge into his or her lifestyle. Perhaps even worse, given the vastness of available unstructured material, the probability of missing relevant health information is increased.

Furthermore, many individuals are not even aware of the relationship between their health and their lifestyle choices. The relevance of health information that appears in the media is not fully appreciated by the individual. For one example, not all individuals are aware of the need for immunization and specific antibiotic prophylaxis when they travel abroad, especially to tropical countries. Given the client-driven nature of content delivery in traditional media, many individuals do not receive specific health related advice, simply because they have not sought information. Thus there is the need for a proactive system that actively ‘pushes’ the content and interaction to the individual and yet be convenient and fit into the individual's existing lifestyle.

Thus, there are unmet needs in the art both for providing relevant, tailored, and understandable healthcare information to individual patients about how their lifestyles affect their health, and for tools individuals can use to exploit the value of such information.

### SUMMARY OF THE INVENTION

Embodiments of the present invention include a system for improving the general personal health of individuals by networking a plurality of individuals who with remote systems, preferably personal computers that are in communication with a central server through the internet. The remote systems also communicate with external servers storing documents relating to health information, non-health information, and entertainment content.

Embodiments of the present invention collect data relating to an individual's demography, personal health, familial health and personalization factors. Using this information, the system generates a health and preferred news and entertainment content profile of the individual. Embodiments thereby provide a healthcare information portal that provides directed healthcare information and other information to attract the individual's attention.

In accordance with further aspects of the present invention, the information and entertainment content accessed by the individual from the personalized portal is screened for related healthcare information by the use of Natural Language Processing algorithms. Accordingly, the individual's profile is modified, and the system modifies the health informational and monitoring content that is applied to a particular individual. Health education is customized to the individual's profile, and the relationship of health to many activities of daily living is explained to the individual. Further, the responses are analyzed, scored and presented to the individual in a variety of reporting formats. In one reporting format, the individual's health status is followed in time and represented on a graph. The health status may also be color coded and represented as such on a calendar. In another reporting format, the individual user is represented pictographically as an image or an animated character, whose appearance and/or actions portray his/her health status. The health status of individuals may also be ranked or graded after comparing it with other users similarly enrolled into the system.

The system also monitors the individual for the development of health conditions that he or she is at a greater likelihood of developing given his/her profile. Health education and monitoring content includes advice on screening for chronic health conditions, cancers, and other conditions with multi-factorial causation.

Additionally, a healthcare analyst or another individual wishing to communicate with individuals enrolled into the system may be granted access to the system. The analyst may utilize the data gathered by the system for the purpose of health research and for marketing consumer products to the individuals. Further, the analyst may utilize the system to communicate miscellaneous health information or news to the individuals.

In an alternative embodiment of the invention, the system consists of a resident program that works in the background of the remote system, scans the information accessed by the individual on the internet, and screens it for health related correlates. Data processing is similar to that with the preferred embodiment. The resident program may also be used to display queries, reports and health information independent of the browser program. Additionally, the information contained within an individual's online daily planner may be synchronized with that on an individual's handheld computing device, such as a PDA.

### BRIEF DESCRIPTION OF THE DRAWINGS

FIG. 1 is a schematic block diagram illustrating the architecture of the system according to a preferred embodiment of the invention;

FIG. 2 is a schematic view of registration document as generated by an embodiment of the present invention and appearing on the remote system of FIG. 1;

FIG. 3 is a flow chart illustrating the various steps included in the method of the invention;

FIG. 4 is a schematic block diagram illustrating the architecture of the system according to an alternate embodiment of the invention;

FIG. 5 is a representative document appearing on remote system of FIG. 1; and

FIG. 6 is a representative report appearing on remote system of FIG. 1.

### DETAILED DESCRIPTION OF THE INVENTION

In the following description, a source of content is understood to include a record of the content, such as an HTML file and a server, respectively, to provide non-limiting examples. Personalized health content is understood to refer generally to health content that is personalized to an individual's profile and general health attributes, and not merely to a health situation of a generic individual having a given health related condition. In a system for delivering health information to, and monitoring a plurality of individuals, personalized health content is understood to refer to health content customized to each individual. The statement that an input device is in communication with some data processing means is understood to mean that the data processing means is adapted to use data specified by the input either directly or indirectly. The term server is understood to refer to an information-generating device capable of communicating with a plurality of clients; servers include computer servers and television delivery systems. The term individual is understood to refer to a person at risk for engaging in behavior having adverse health consequences, as well as a person suffering from a chronic condition or disease.

An exemplary embodiment discussed below describes a computer-based implementation of a system and method for general personal health improvement with special emphasis on physical activity, diet and nutrition. It will be clear to an individual with ordinary skill in the art that the present invention is suitable for preventive care directed to many other health conditions. Moreover, there are many well-known structures, interfaces and processes that are suitable for implementing the present invention.

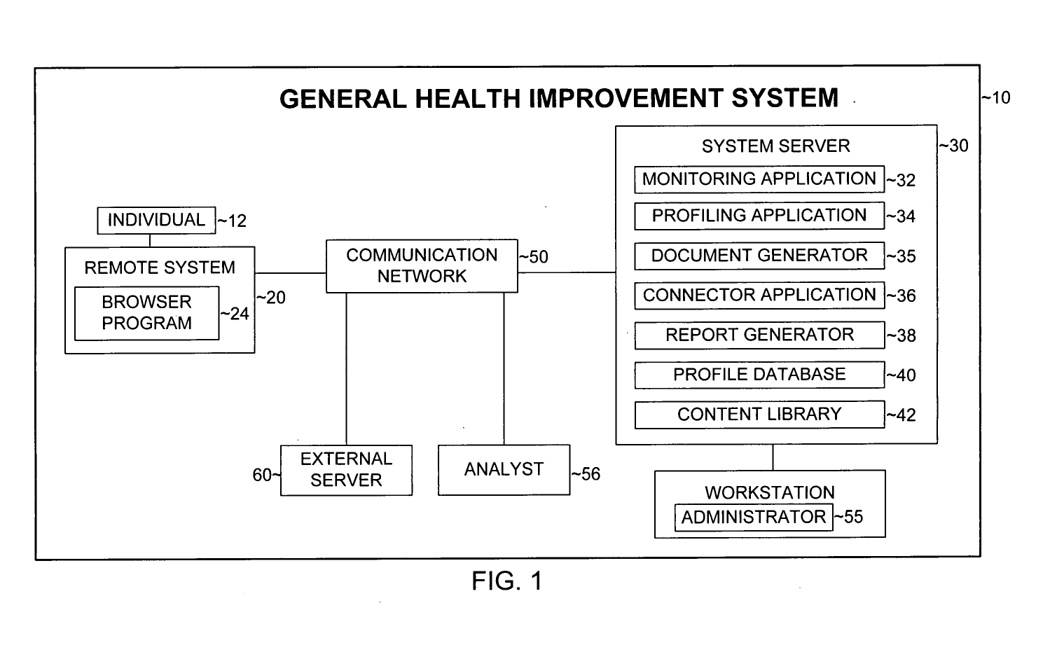

FIG. 1 illustrates the architecture of a general health improvement system 10 according to a presently preferred embodiment of the present invention. A system 10 is used by a individual 12 whose health situation is monitored utlizing remote system 20 and communication network 50 to transfer and receive information from a server 30. The remote system 20 suitably is a personal computer with browser program 24 for accessing an information portal to be described below

The browser program 24 is an Internet browsing program such as Microsoft Internet Explorer® available from Microsoft Corporation, Redmond, Wash. The communication network 50 suitably is a secure Internet connection. For the sake of clarity, only a single individual 12 is shown in FIG. 1. However, it is to be understood that in the actual implementation of the system 10, server system 30 is in communication with a plurality of individuals 12.

The server 30 includes a monitoring application 32, a profiling application 34, a document generator 35, a connector application 36, a report generator 38, a profile database 40, and a content library 42. An administrator 55 preferably utilizes a network workstation to review and update the content and other software components of the server 30.

Additionally, access to the system 10 may be granted to an analyst 56 at the discretion of the administrator 55. The analyst 56 may be any person who wishes to communicate with the individual 12. The analyst 56 also suitably is a public health researcher who wishes to enroll individuals into a new lifestyle modification program, or a representative of a company that wishes to identify individuals who could benefit from a new drug, a home-test kit, nutritional supplement, workout equipment or other health related item or system.

The individual 12 may additionally be in communication with one or more external servers 60 over a communication network 50. The external servers 60 contain documents such as HTML files or other sources of information that relate to health, entertainment or other content. For one non-limiting example, a page-based implementation suitably includes documents including home pages of companies or organizations such as health related not-for profit organizations, Internet directories, and pages containing weather news, sports news, stock quotes, or other information of interest or potential interest to the individual 12.

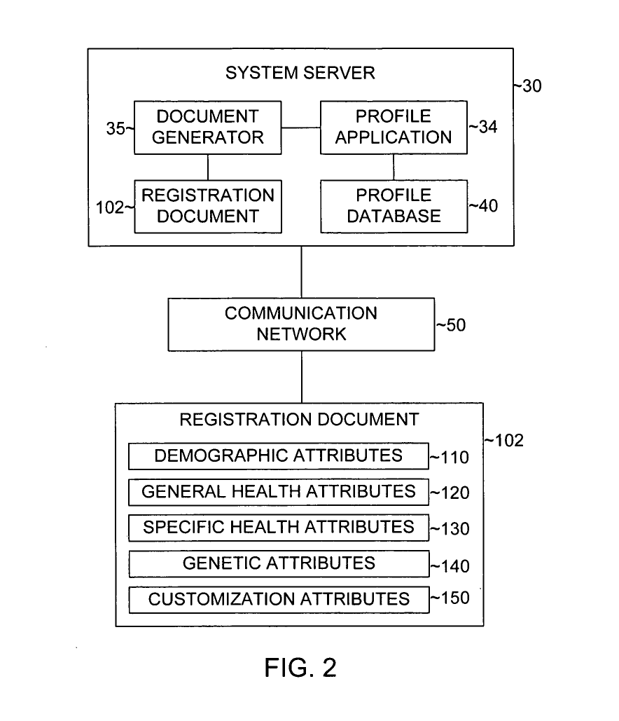

Referring to FIG. 2, document generator 35 is capable of generating an interactive document 100 and transferring the interactive document 100 through the network 50 for display to the individual 12 at the remote system 20. The document 100 suitably is in the form of an HTML file having embedded JavaScript or VBScript which dynamically responds to information entered into its data entry fields. At the time of initial registration of the individual 12 into the system, the document generator 35 generates a registration document 102. The registration document 102 includes queries to be answered by the individual 12 in a number of categories. The categories suitably include demographic attributes 110, general health attributes 120, special health attributes 130, genetic attributes 140, and customization attributes 150, each of which are further described below. Other queries directed at healthcare issues, such as the sexual preference or sexual practices of the individual 12, also can be incorporated in the registration document 102. As will be appreciated, the individual 12 may be reticent to answer some queries which he or she finds to be too personal in nature. In such a case, the individual can choose not to respond to some of the queries without undermining value of the rest of the interaction between the individual and the system 10. All the information that is inputted into the document 100 and the registration document 102 is stored within a profile database 40.

The demographic attributes 110 suitably target the name, age, sex, marital status, income, race, ethnicity, and other demographic information about the individual 12.

The general health attributes 120 suitably target physical attributes of the individual 12, such as weight and height, as well as lifestyle attributes such as level of physical activity, type of diet, smoking and alcohol consumption status, sexual preference and/or sexual practices, illicit drug use, and other such data. The status of an individual with regard to general health attributes 130 can be used by the system 10 to determine the level of risk faced by individual 12 in developing a given lifestyle related disease.

The special health attributes 130 suitably target the medical history of the individual 12, including whether the individual 12 is suffering from diabetes, hypertension, coronary heart disease, hyperlipidemia, frequent injuries, psychiatric medical conditions, etc. It also targets whether the individual 12 is on any medication, has had any previous hospital admissions, has undergone screening tests for any diseases or chronic health conditions ranging from alcoholism to cancers, and other special health concerns.

The advantage of including queries regarding the special health attributes 130 is that the monitoring application 32 views the replies of individual 12 in the context of the pre-existing health condition of the individual 12. Replies to standard queries, and the general health improvement solutions offered by the system vary depending on the health condition from which the individual 12 may be suffering. For instance, the type and amount of exercise that is ideal for an individual who is suffering from coronary artery disease is significantly different from that of a normal individual. Similarly, if an individual who is diabetic inputs he or she lately has been experiencing episodes of increased sweating after even mild exercise, the sweating can be flagged as a first sign of potential heart disease.

The genetic attributes 140 target the medical history of the family of the individual 12. Incidence or risk of many chronic health diseases, health related conditions, and cancers are hereditary. For example women who have inherited the BRCA-1 gene are at a greater risk of developing breast cancers. Similarly, in some families, individuals are at a greater risk of developing atherosclerotic plaques and with a subsequent increased risk of heart attack and stroke. Similarly, individuals who are at greater risk for developing heart attacks than the general population due to genetic factors such as familial hyperlipidemic syndromes are more likely to have double or triple-vessel disease, and multiple plaques within a single coronary artery than the individuals without these factors. These individuals are also less likely to present other risk factors for coronary artery disease.

In cases where there is a strong genetic indicator, there is a tendency for the affected individual to be younger at the time of diagnosis of disease, to have multi-centric disease, and to have fewer concurrent risk factors than in other people. For example women with the BRCA-1 gene are diagnosed with breast cancer at an earlier age than the average.

Thus, the genetic attributes 140 target whether there is a history of chronic health diseases, such as diabetes, hypertension, and other diseases, or risk factors of chronic health diseases, such as hyperlipidemia syndromes, obesity, addictions, and other diseases. The genetic attributes also target possible early onset of some cancers among family members of the individual 12.

Therefore, when individual 12 inputs information of a close relative having developed an illness with a known genetic component, he or she is further queried about the age at which the relative developed the illness, the severity of the illness, and the presence or absence of other concurrent risk factors in the relative. The individual 12 is also queried as to whether any other close relatives have developed identical, similar or associated health conditions. This information is used to determine whether there is a significant genetic risk in the individual's family. Risks associated with genetic factors may be further quantified by the severity of the risk, the number of close relatives having developed the disease, and the severity of the disease in the afflicted relatives.

For example, if the mother of the individual 12 has developed breast cancer at the age of 30, then there is a high likelihood of it being related to familial factors such as the BRCA-1 gene. The importance here is that the recommendations for cancer screening in the individual 12 is different from that in persons without the particular aspect in their profile. The index of suspicion for cancer would also be significantly higher in such an individual 12. Similarly, if the individual 12 has a sibling who had a heart attack at the age of 35, he or she may be advised to have a screening for hyperlipidemia.

The customization attributes 150 target the preferences of individual 12 relating to the appearance and preferred medium of presentation of content such as videos, pictures, and hyper-text links to external servers 60. The customization attributes 150 also suitably are used for determining the individual's motivational drivers such as the importance that individual 12 places on physical appearance, longevity, quality of life, family life, social acceptability, social responsibility, economy, and other factors. Additionally, the customization attributes 150 suitably includes queries that are used for determining the individual's comprehension capacity, including language skills, reading habits, educational background, and related details.

Once the individual 12 has responded to queries directed to discern information about the demographic attributes 110, general health attributes 120, special health attributes 130, genetic attributes 140, and customization attributes 150, replies to queries are transmitted by remote system 20 to the profiling application 34 on the server 30. The profiling application 34 utilizes the data contained in the replies to generate the profile database 40 which stores the information relating to the individual 12. The monitoring application 32 references the profile database 40 to glean information about the individual 12 to determine the health education, monitoring and general health needs of individual 12. As a result, the application 32 suitably suggests to the individual 12 one or more services to which the individual might subscribe or programs in which the individual 12 might enroll. For one non-limiting example, the service may be a combination of weight loss programs, physical fitness programs, health educational programs or may simply be a general personal health improvement program, depending on the health needs of the individual.

It is known to persons of ordinary skill in the art how to write a computer program that automatically generates personalized portals on the basis of user preferences. Methods to automatically create internet page documents that contain user-requested content in addition to unrequited content, such as advertisements, relating to the user's interests is well known in the art.

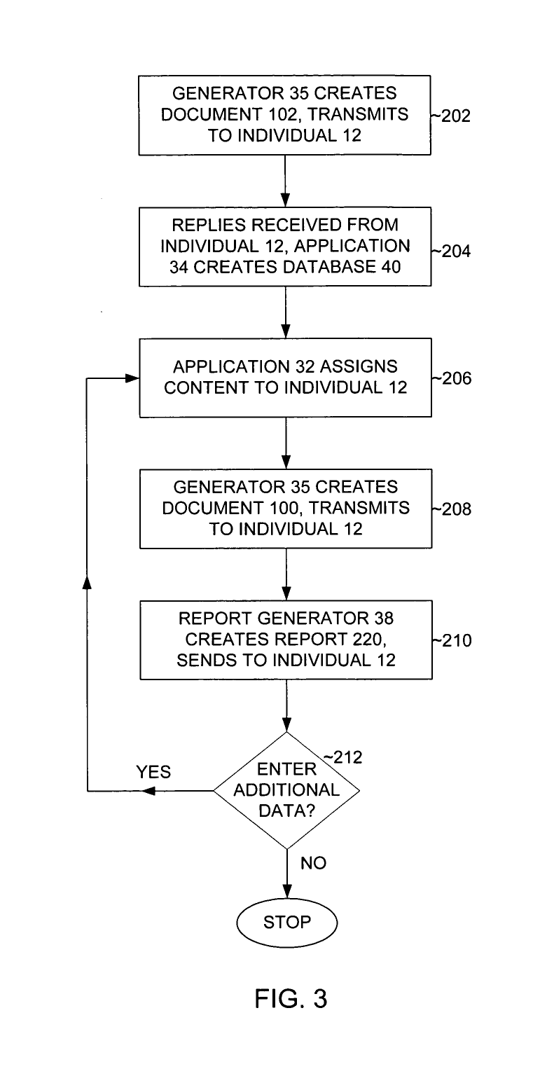

FIG. 3 is a flowchart providing an overview of the various steps involved in the system-individual interaction. At a block 202, the document generator 35 generates a registration document 102 and sends it to the individual 12 over the communication network 50. At a block 204, the profiling application 34 receives the replies of the individual 12, and uses it to create the profile database 40 for the individual 12. Additionally, the profiling application 34 may use the received data to suggest to the individual 12 a health improvement plan in which the individual 12 might enroll.

At a block 206, the monitoring application 32 references the profile database 40 of the individual 12 and assigns content to the individual 12. Additionally, content may also include that which is assigned to individual 12 by the administrator 55 and/or the analyst 56. The content includes educational materials, queries to be answered, and other materials of general interest to the individual 12. The content primarily is drawn from the content library 42 located on the server 30. Additionally, content from the external server 60 may be also be assigned to individual 12.

At a block 208, document generator 35 creates documents 100 for transmission to the individual 12. The documents 100 preferably are HTML documents with embedded pictures, streaming video, audio files, and other substantive content. When required, the document generator 38 references material on the external server 60 in order to create the document 100. Alternatively, the document generator 35 adds a hypertext link documents and files located on the external server 60. Alternatively, the document generator 35 may include embedded code instructing the Internet browser program on the remote system 20 to download and display documents and files from one or more external servers 60.

At a block 210, the report generator 38 creates a customized report 220 that provides feedback to the individual 12 regarding his or her status of health. At a block 212, the individual 12 is given the option of inputting additional data into the system 10. If the individual 12 chooses to input additional data, the program continues to the block 206, and a new interview document is provided. If the 12 individual chooses otherwise, the session is terminated.

In the example described above, multiple queries covering a range of health-related topics are included within a single registration document 102. However, in an alternative embodiment, the queries may be distributed over multiple registration documents 102. The registration documents 102 suitably also elicit general information about what forms of entertainment or news content are of interest to the individual 12. Additionally, queries may also be spread out in time in such a manner that the content presented to the individual 12 is an immediate function of the query put to the individual 12.

For one non-limiting example, the individual 12 may be queried as to whether he or she has a long-standing mole or scar that has recently changed in size, shape or color. If the individual 12 replies in the negative, the individual nonetheless may be informed that this query concerns what may be the first sign of a common cancer, namely malignant melanoma. On the other hand, if the individual 12 replies in the affirmative, he or she may be asked further queries to establish whether the individual 12 needs expert evaluation by a healthcare professional.

Another advantageous aspect of an embodiment of the present invention is the provision of other content related to an individual's interests. Many internet companies, such as Yahoo! Inc.® offer news and entertainment content that is personalized to individual users, offering a personalized webpage or ‘personalized portal,’ the user initially selects from a list of topics of interest to the user. Subsequently, a computer program selects the latest news and entertainment content relating to the topics of interest and presents it to the user on an automatically generated webpage or portal. ’ Often, the headline of a news or entertainment story is presented to the individual as an internet link to the complete story. The personalized portal typically contains additional internet links to other services provided by the internet company, including internet search functions, online daily planners (such as a Yahoo! Calendar), links to stock quotes, internet based email, short text messaging, weather news, automobiles, travel related information, and other forms of information.

In one embodiment of the invention, information and entertainment content preferences of interest to the individual 12 are collected and presented to the individual along with personalized health-related content. In this embodiment, the personalized portal includes news and entertainment content in addition to the general health improvement content of system 10. Advantageously, because the system is pro-active, the system 10 would present general health improvement content to the individual 12 without him or her having to seek the information. Further advantageously, the system 10 suitably is configured using “cookies” or other recognition protocols which would allow a user to access personalized content without having to manually log in each time the user accesses the system. As will be appreciated by those ordinarily skilled in the art, this automatic access could be controlled by user options so that the individual 12 can prevent others from seeing his or her information time if he or she accesses the system 10 from a publicly accessible or otherwise shared computer.

Advantageously, another aspect can be included in an embodiment of the present invention by which general Internet content accessed by the individual 12 may be parsed for correlations with health-related issues. In this case, the system 10 monitors the nature of the information and entertainment content requested by the individual and may provide general health improvement and health educational content related to the content sought by the individual along with the related requested content.

For one non-limiting example, many individuals who travel abroad make travel and accommodation reservations online from web pages that are accessed from Internet links on the personalized portal. In addition to providing the requested content, the system 10 additionally provides the individual 12 with links to Internet pages containing travel-related health advice. The travel-related health advice suitably includes U.S. Government travel advisories regarding current disease outbreaks. The travel-related health advice also suitably includes standard recommendations on vaccination and prophylaxis for certain diseases, such as vaccinations for yellow fever while traveling to Africa, chemoprophylaxis while traveling to nations where malaria is prevalent, and similar types of cautionary information. The system 10 also might offer general health related precautionary advice, such as suggesting to the individual 12 that he or she consume only bottled water in order to prevent gastrointestinal distress.

In addition, individuals who seek local maps, tourism related information, and information on the local weather of a certain geographical location may simultaneously be provided with the standard travel and/or healthcare-related recommendations, or links to websites providing the same. An advantage of the system 10, to name one example, is that, since the information is automatically provided, the individual 12 suitably is made aware of the fact of how his or her interests and queries have a relation to his or her health. On the other hand, if the health content merely was provided upon request by the individual 12, many an individual 12 would not have received the information because the individual 12 may have been unaware of the relationship of an activity to one's health.

This information-correlating feature of an embodiment of the present invention is not limited to the case of the individual 12 searching for travel information. There are many examples in which correlation of healthcare-related information could be performed to benefit the individual. To name another non-limiting example, if the individual 12 browses the Internet for information prior to purchasing a new automobile, the individual 12 may be provided with additional information on the standard features and safety related options that may be considered while making such a purchase. Similarly, statistics on relative safety of different types or makes of automobiles could be offered to the individual 12 for consideration. For another non-limiting example, if the individual 12 browses the Internet for personal computers or televisions, the individual 12 could be presented with information on the healthcare advantages of using low-radiation, flat-screen monitors, such as liquid crystal or plasma displays.

Other advantageous aspects can be included in embodiments of the present invention. The ability to link a personal digital assistant (PDA) to the store of information controlled by the personalized portal maintained for the individual 12 on the system 10 allows for further application of healthcare information. An on-line calendar or daily planner can be synchronized with a similar application on a PDA, making healthcare information and applications portable. Using a PDA, the individual 12 may set alerts for himself or herself on the PDA, so that the individual 12 is reminded to undertake a health related activity, such as exercising at a regular time in the day. Similarly, in the specific instance of weight loss, the individual 12 may be posed queries by the PDA that relate to his or her adherence to a diet. The replies entered by the individual can be transmitted to the system 10 in the same manner as is familiar to users of PDAs and computers, allowing synchronization of the PDA information with that stored in the online planner resident on the system 10.

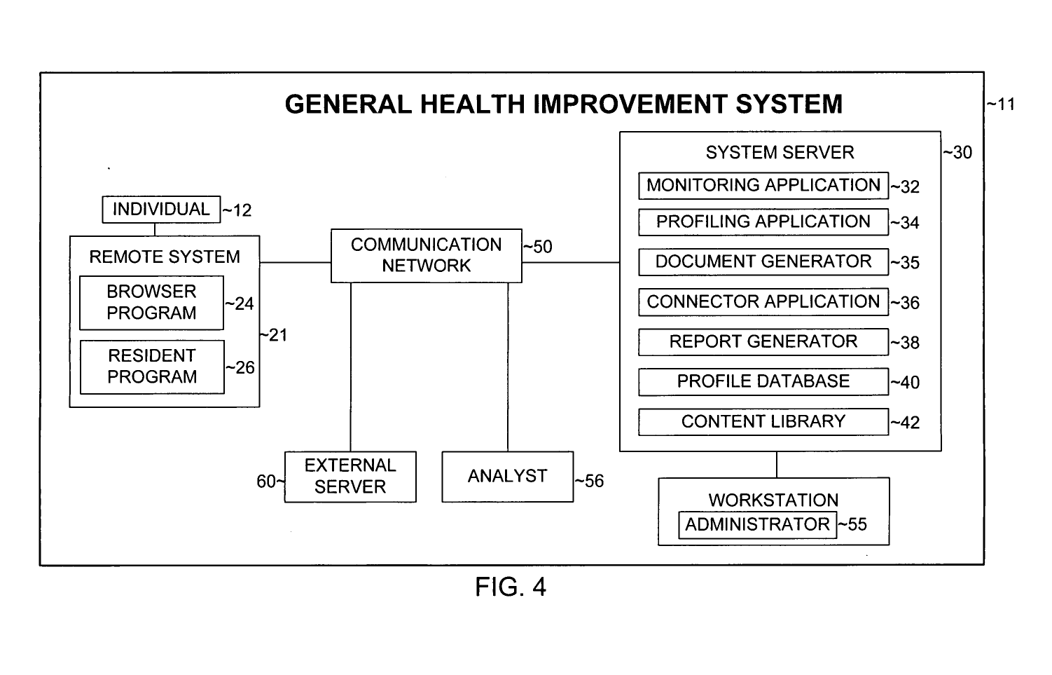

FIG. 4 is a schematic block diagram illustrating the architecture of a system 11 according to an alternate embodiment of the invention. Elements of the system common with the system 10 (FIG. 1) are labeled with identical reference numerals, and the operation of the common elements will not be repeated here in the interest of brevity.

The system 11 shown in FIG. 4 is similar to the system 10 of FIG. 1 except that the remote system 21 in the system 11 supports a resident program 26 in addition to the browser program 24. The resident program 26 is a software program that works in the background of the operating system of a system 21, and monitors the nature of the Internet content accessed by the individual 12.

The resident program 26 uses standard natural language processing algorithms to search for health related information correlated with the Internet content sought and/or accessed by the individual 12. When the resident program 26 encounters any administrator-defined correlated health-related information, it presents the information to the monitoring application 32.

Advantageously, in the system 11 all internet content accessed by individual 12 is scanned for relevant health-related information. Therefore, to name one non-limiting example, whether the individual 12 accesses a travel site through a link on the personalized portal or by directly accessing the travel site, the system 11 can seek and present relevant healthcare-related information. How to create software operable to analyze information sought or displayed is known to those ordinarily skilled in the art.

An advantage of the embodiment of the system 11 is that the individual would not need to log into the system 11, or use a personalized portal as a gateway to the Internet to obtain relevant healthcare-related information. Because the resident program 26 monitors Internet access through the system 11, healthcare-related information relevant to sought or displayed content can be sought for any user of the system 11

Online features of the personalized portal may be incorporated into the resident program. For instance, resident program 26 may contain a standard, built-in daily planner that allows the individual 12 to input information and synchronize it with the online planner. Further, the information contained within the standard planner may be simultaneously synchronized with an individual's PDA.

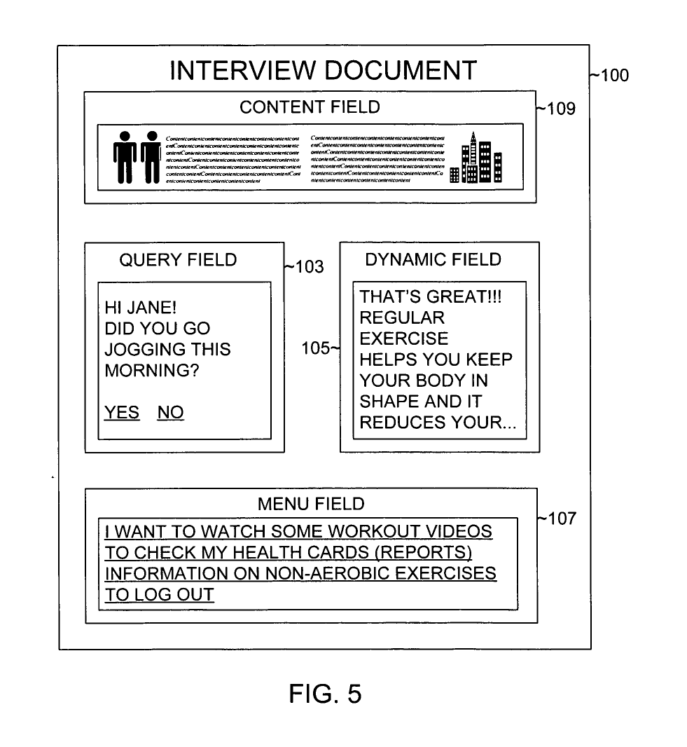

FIG. 5 is a schematic view of an interview document that appears on the display unit of remote system 20. A content field 109 lists news, information and entertainment content that suitably is selected to personalize the portal for the individual 12. The content field 109 may additionally include multimedia content, such as streaming videos, audio, pictures, advertisements and miscellaneous third-party content.

A query field 103 contains of or more queries, one or more possible replies to the queries, and data entry text fields on the health and other profile related variables of the individual 12. For one non-limiting example, the individual may be asked whether she went jogging that particular morning. The possible replies are ‘YES’ and ‘NO’ in the form of hyperlinks, with pre-defined actions associated with each of the possible replies. The pre-defined actions associated with each of the replies can include a type of content that appears in any of the remaining fields, a series of changes to be made in amending/appending information in the profile database of the individual 12, or other actions. Accordingly, when the individual 12 chooses one of the possible replies, such as by clicking on the reply with a cursor controlled by a computer mouse, the content on in the query field 103, the dynamic field 105, the menu field 107 and the content field 109 suitably is changed to reflect the possibly reply chosen by the individual.

The menu field 107 contains one or more hypertext links that allow the individual 12 to navigate within the system. For one non-limiting example, the individual 12 may choose specific content, such as viewing multimedia content, such as an exercise video, review his or her health reports for a specified period of time, obtain more information on a topic of interest, log out of the system, or take other actions.

As is illustrated in FIG. 5, the query field 103, the dynamic field 105, the menu field 107 and the content field 109 are shown as constituent elements within a single document. However, many variations and relative arrangements of the display fields, and methods to display the fields on the remote system 20 are possible. For instance, the dynamic field 103 could be in the form of a window that pops up in response to a specified action of the individual 12. Alternatively, any of the fields 103, 105, 107 and 109 suitably can be displayed as a new window on the browser 24. Similarly, multimedia audio and visual content may be displayed through the use of separate software, such as Windows Media Player® available from Microsoft Corporation, Redmond, Wash.

It will be understood by one ordinarily skilled in the art that HTML documents may be so generated that the internet browser on remote system 20 accesses third-party content, including that available from external server 60 directly through the internet. An advantage of the above is that all the content, including the advertisements need not be physically located on system server 30. An added advantage is that, since individual 12 obtains the content from external server 60 directly; there would be no possible copyright violations by administrator 55. In addition, individual 12 is provided additional access to a greater range of material without administrator 55 having to actually create the entire content.

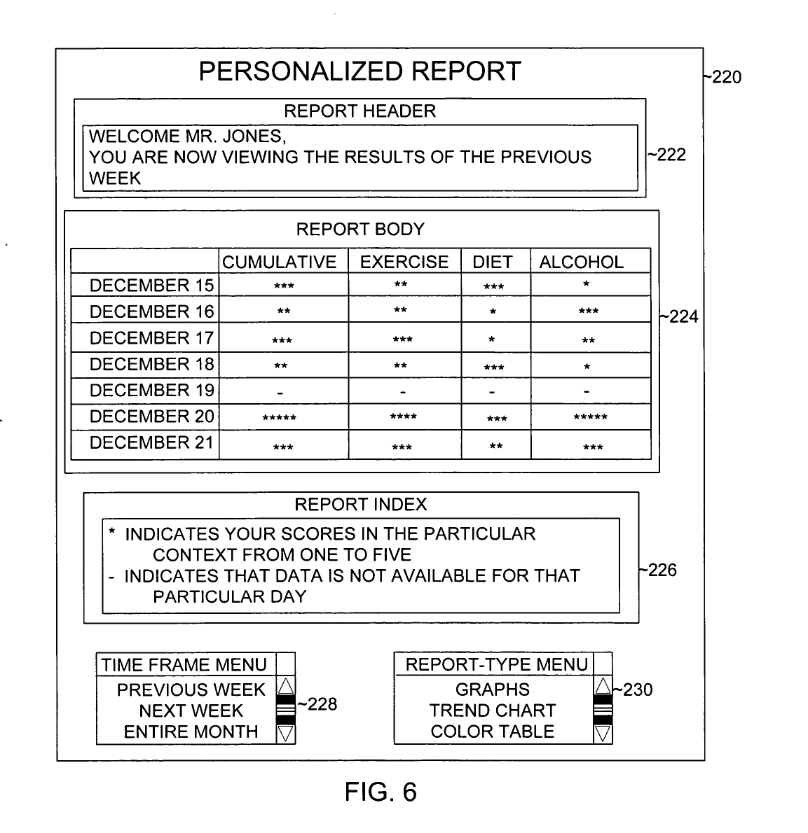

FIG. 6 is a schematic drawing of a personalized report which is one type of report that can appear on the remote system of FIG. 1. Reports are important in that they serve as an accountability mechanism for the individual 12 within the system 10. Further, reports provide valuable feedback to the individual 12 regarding his or her health status for the time frame under consideration. Although a preferred structure of the report 220 in terms of drop down menus and other features are described, it will be appreciated that other formats, layouts, and structures also suitably can be used. For one example, instead of a drop-down menu, a pop-up dialog window could be used. Embodiments of the present invention are not limited by screen formatting or presentation choices.

The personalized report 220 includes a report header 222, a report body 224, a report index 226, a time-frame menu 228 and a report type menu 230. The report header 222 includes a greeting to the individual 12 and introduces the report. The report body 224 provides the individual's health status information in a preferred format and for the time frame desired by the individual.

The report index 226 contains an explanation of the report 220 in simple terms that may be understood by the individual 12. Additionally, the report index 226 may contain feedback and comments of the administrator 55. The time-frame menu 228, which suitably is a drop down menu, that allows the individual 12 to obtain the reports for a different frame of time such as for the previous week, for an entire month, etc.

The report type menu 230 also suitably is a drop-down menu that allows the individual to obtain a report in a different format such as a health graph, health trend-chart, health image map, color-coded table depicting the level of risk, etc.

As shown in FIG. 6, the individual 12 receives a report that scores the healthfulness of the individual's behavior for a period of time. Quantitative analysis of the information supplied by the individual 12 and calculated by the system 10 suitably is used to provide concrete information to the individual 12 for measurement, motivation, and other purposes. Advantageously, motivation can be drawn from evident, quantitative representation of progress made by the individual 12. Potentially even more advantageously, representation of the progress made by the individual 12 relative to other similarly-situated persons taking part in a comparable program promotes adherence with healthful diet, exercise, and other regimens. Embodiments of the present invention make this information available to the individual 12 using the system, as is further described below.

Methods of Data Analysis and Presentation to the Individual-Risk Calculators, Scoring and Ranking

Risk may be presented to the individual in many formats. Risk factors of developing certain chronic health conditions may be divided into two self-explanatory categories: modifiable and non-modifiable risk factors. For example, the non-modifiable risk factors for developing coronary artery disease may include the age of the individual 12, whether the individual 12 is male, whether the individual 12 is Caucasian, whether the individual is a post-menopausal female, and whether the individual 12 has a family history of the disease. Modifiable risk factors may include obesity, smoking, alcohol consumption, level of control over diabetes, hypertension, and exercise and dietary habits.

Healthcare providers and public health specialists primarily focus on the modifiable risk factors since there is little that may be done about the non-modifiable risk factors from the medical point of view. However, non-modifiable risk factors are also important in that they give a good idea about the baseline level of risk in a particular individual in whom all the modifiable risk factors have been controlled.

The level of risk in an individual with two or more risk factors is the product of the level of risk levels of the individual-risk factors. Thus, the level of risk for an individual who has an unhealthy lifestyle increases greatly with two or more risk factors, For example, for an individual 12 who is a hypertensive, smokes and has a sedentary lifestyle, the risk increases significantly. In this case, the data may be presented to the individual 12 as the risk faced by the individual 12 of developing one or more diseases, in relation to the baseline risk for a hypothetical person who has no such modifiable risk factors. The level of risk attributable to various health related activities and lifestyle factors is widely available in the medical literature and understood by those having ordinary skill in the art.

For another example, in the case of the individual 12 who is hypertensive (risk of mortality increased a times), smokes (risk increased b times) and has a sedentary lifestyle (risk increased c times), the net modifiable risk, r=a*b*c. Similarly risk states may also be computed for the individual with relation to all factors, whether the factors are modifiable and/or non-modifiable. Presenting data in such a quantifiable form advantageously shows the individual 12 identifiable and quantifiable health related benefits of modifying behaviors and adopting healthier lifestyles. As the individual 12 modifies his or her lifestyle-related behaviors, the modifiable risk factor, r may be recomputed to reflect the new behaviors.

Because most health related behaviors are related to more than one disease state, the risk factors, r may be computed for more than one disease state, and presented to the individual. The r values may also be turned into a health score, s by multiplying its reciprocal with a suitable whole number such as 10 or 100:
s=x*(1/r), where x is any whole number, such as 10 or 100.
2hen x equals 100, s is expressed as a percentage.

Similarly, one may also compute health scores by a formula of the type,
s=p+{q*(1/r)}
where p and q are constants and the sum of p and q is a whole number, x. Many other methods and mathematical formulae may be used by the system to compute s values, which are preferable in specific circumstances.

Health scores may be preferred to r values by many individuals since; unlike r values, a greater health score indicates a better state of health for the individual. Risk factors such as the r values may also be computed for a certain interval of time, such as for a day, a week or on a monthly basis. For instance, individuals wishing to lose weight may be assigned certain risk values for each of a range of health related behaviors. For example, a risk factor a may be used to represent not exercising the required amount, a risk factor b may be used for having eaten high energy foods, such as dessert, etc. The health scores are then computed for the individual 12 for an interval of time, and presented to the individual 12 in addition to the individual's previous health scores.

An objective measurement of the success of the plan, such as the actual weight lost by the individual over the period of time may be additionally included in deriving the final scores.

In addition, the individual 12 may also be provided with grades or a rank with regard to other individuals who are following a similar plan. Percentile scores and grade point average (GPA) scores may also be provided to the individual. Providing relative scoring, such as percentile scores or ranks, advantageously motivates the individual 12 to abide by the plan. Further, the individual 12 may also be allowed to trace the scores of other similarly placed individuals, and compare himself or herself to others.

Though the ranking system described above does not contain safeguards to prevent cheating by individuals, the expectation is that most individuals who are sufficiently motivated to enroll into the system would be reluctant to misrepresent facts in order to obtain a better ranking in an essentially anonymous system. Further, individuals who enroll into the system may be given the option of being excluded from the ranking system at any point of time.

Additionally individuals may be given the option of communicating with other individuals who are enrolled into the system by posting messages on internet bulletin boards. Methods to create internet bulletin boards and allow individuals to post messages on these bulletin boards are well known in the art.

Health Graphs

Risk scores may also be represented to the individual 12 as a graph by tracing the risk factors over time. Health graphs are advantageous in that they are concise, simple to understand, and employ a familiar format. Health graphs may additionally contain the health tracings of other individuals, thereby allowing the individual 12 to compare his or her progress with others following a similar plan.

Health graphs may additionally contain the tracing of a hypothetical ‘average individual’, one whose scores are the arithmetic average of all the individuals following a certain plan at a certain point in time. The advantage of depicting the general health status scores of other individuals is that the motivation of the individual to abide by the plan is directly increased as a result of the ‘competition’.

Health Color Coding

In health color coding, the health scores of individuals are coded whereby each color represents a different level of health of the individual. For instance, the color red may represent a poorer state of general health or risky behavior, while the color green or blue may represent a healthier state. Similarly, the level of health of the individual 12 may be represented by different shades of gray in a black and white context.

The color assigned to the individual for any particular period of time is a direct function of the health scores, s. For example:
IF s≧85, THEN BACKGROUND COLOR=“BLUE”
ELSEIF s≧60 THEN BACKGROUND COLOR=“AMBER”
ELSE BACKGROUND COLOR=“RED”
   The advantage of health color coding is the simplicity of representation of information to the individual. An added advantage is that health color coding information may be integrated with the individual's daily planner and presented to the individual. For example, the background color of the individual's daily planner may reflect the individual's general health status for that period of time. Similarly, the color coding information for a period of time may be presented on an online calendar.
   Health Graphics

Health graphics are visual representations of an individual's health condition in the form of images or video graphics of characters that represent the individual 12, and responds dynamically to the individual's inputs.

The general health status of the individual 12 is depicted by projecting it directly on a customized image of a body. The individual's general health attributes 120, specific health attributes 130, and genetic attributes 140 are applied to a generalized health model to generate a personalized model of the individual 12, which comprises an HTML file encoding an image map of a body.

The formatting of the body image is customized to the individual 12, such that he or she identifies with the body image. A set of the characteristics of the body image matches a set of predetermined characteristics. In particular, body image characteristics preferably match demographic attributes 110 and/or physical characteristics chosen by the individual. Such characteristics include age, height, gender, weight, build, skin color, hair color, and, optionally, identity of a fictional character. The body image may be a schematic figure representing the individual, a photographic reproduction of the individual's appearance, a representation of a cartoon or fictional character, or a representation of a character in a field of interest of the individual such as a favorite basketball player or movie actor, or another such representative visual image.

Health graphics are particularly valuable while presenting health information to the younger individuals, particularly those who have not yet reached the age of 18. In addition to body images of characters, video graphics may also be used to display the personalized health status of the individual. For instance, the level of activity ‘energy’ of the graphics character may be proportional to the health status of the individual. Alternatively, a healthy character may be represented by a brightly appearing character on the screen, which reduces in intensity with deteriorating health status.

In health graphics, graphical representations of other individuals who are enrolled into the system may be additionally represented on the display unit of remote system 20. For the sake of clarity of understanding, some examples of the health graphics method of display are described below. However, it may be noted that there are many other possible combinations and instances wherein the health graphics methods may be used to display personalized health information. The following represent some non-limiting examples of graphical information that might be displayed:
1. Energy Levels—A general level of activity of the individual 12 can be graphically portrayed. For example, if the individual 12 inputs into the system 10 that he or she has not undertaken any physical activity for a period of time, the individual may be represented by a character that is slow-moving with a weary look on its face. On the other hand, if the individual 12 is or has become more active, or other persons with whom the individual is being compared are more active, those persons will be represented by more lively, happier looking characters.
2. Body Image Changes—A general appearance of the individual 12 can be graphically displayed. If the individual 12 indicates that he or she has not adhered or is not adhering to a diet, a character representing the individual 12 may be shown with a rounder, flabbier body image and may be shown as increasingly more so over time. On the contrary, the body image of the individual 12 who reports successful adherence to the diet or loss of weight can be displayed as more fit-looking. Similarly, representations of other persons with whom the individual is being compared can be similarly represented as appropriate to their situations.
3. Body Component Changes—Instead of graphically representing the entire body image, it may be desired to represent only a relevant part or organ of the body of the individual 12. For example, smokers suitably are depicted as having darker lungs than non-smokers. Additionally, smoker characters on the display may also be shown to cough intermittently. In addition, the baseline energy level of smokers is considerably lesser than that of their non-smoking peers. Other examples include representing clarity of arteries where cholesterol or body fat is a problem, condition of a liver in a person who consumes alcohol, other similar bodily attributes.
4. Body Mapping Scoring—Quantitative scores may be provided for each of the individual's organ systems and body components, such as liver, lungs, heart, eyes, teeth, and other bodily aspects on the basis of the initial profile submitted by the individual 12. The scoring system is mapped to parts of the individual 12 and may be associated with a character graphically representing the individual as previously described. Different organ systems and body components are represented by respective anatomical location on a map. When the individual clicks one's computer mouse over a particular body location of the character, the system provides the individual 12 with his or her scores for the respective organ system. The individual 12 is additionally provided information on how to improve one's rating for the particular organ system. For example, when a smoker clicks on the lungs area on the character, the system informs the smoker that the lungs are black in color as a result of the deposited tar in the smoke. The individual 12 may be further informed of the other ill effects that are a result of his or her lifestyle choices, such as lack of physical activity, poor eating habits, substance addictions, and other factors.
5. Follow-Up Queries/Dynamic Updating Of The Character's Image Map/Graphical Feedback And Encouragement—Whenever health information has been provided to the individual on a particular organ system, the individual is queried regarding behavioral change, after a period of time. In case of the individual's having incorporated the healthy behaviors into his or her lifestyle, the appearance of the character is changed to reflect the present situation. For example, a young individual may be queried whether he or she brushes his or her teeth every night before going to bed. If the individual 12 replies in the affirmative, the teeth of his or her online character start to sparkle. If the individual 12 who replies in the negative, a ‘cool’ appearing character with which the young individual can identify himself or herself appears and tells the individual 12 that it is necessary to brush one's teeth before going to bed. This may be followed up a few days later by the same query, and if the reply is in the affirmative, the teeth of the young individual's character start to turn white.
   Weight Reduction and Personal Fitness Program

In a weight reduction and/or fitness program, the individual 12 initially registers with system 10 through the use of registration document 102. The profile application 34 generates an initial profile database 40 for the individual 12 from the individual's responses. The monitoring application 32 scans the individual's profile database 40 and determines the appropriate program for the individual 12.

An individual 12 enrolled in the program is given sets reasonably achievable targets and health related goals for him/herself. Alternatively, the individual may decide to follow one of a number of generic plans available on the system.

Health related goals relating to dieting may include refraining from having snacks while watching television, giving up post-supper desserts, using artificial sweeteners instead of sugar in one's coffee, having diet colas, and other similar behaviors. The individual 12 may also be provided with standard diets with possible dietary substitutions. Additionally, the individual 12 may also plan a diet for himself or herself on the basis of his/her preferences and buy using a diet calculator incorporated in the system 10.

Health related goals relating to physical activity may include, for example, jogging a certain distance everyday, or exercising for a certain fixed amount of time. The individual may be allowed to choose the kind of activity, such as swimming, jogging, aerobics, or other forms of exercise that he or she wishes to undertake and the amount of the activity.

Each one of the health related goals that the individual 12 agrees to fulfill is assigned a numerical value, and the individual's score for a period of time is an arithmetic function of the individual scores. On continued follow-up, queries are put to the individual 12 to determine the extent to which he/she is following the prior agreed-upon plan. The closer the individual 12 is to following the plan in its entirety, the greater the individual's score.

Further, the individual 12 is asked to input his or her measured weight after regular, fixed intervals of time. This serves as an objective measure of the success of the plan in the particular individual. The health scores of individuals may additionally incorporate this objective criterion to increase the accuracy, and correlation coefficient of the scoring system.

In addition, the incorporation of subjective as well as objective means into the scoring system allows the administrator 55 and the analyst 56 to determine the concurrence, correlation and accuracy of the non-objective scores derived from the individual's responses to queries to the objective result, which is the actual weight lost by the individual 12. This has the added advantage of allowing the administrator 55 to fine-tune the scoring system.

To further support the individual's efforts in reaching his or her healthcare goals, the system 10 suitably parses the individual's computer activities to identify, collect, and present relevant healthcare information. For example, if the individual downloads the local map of another town from the Internet, the system 10 may present a list of healthful restaurants or shops automatically or if the individual 12 indicates he or she is traveling to that town in response to a query from the system 10. The individual 12 suitably also is provided with the location of health clubs and gymnasiums in the local area. The locations identified by the system suitably may be depicted directly on the local map.

If the individual 12 chooses a generic plan, the individual 12 advantageously has access to a peer-group of individuals with similar problems. This peer group of individuals serves as a support system for the individual 12. Given that individuals are ranked, given grades and percentile scores within the group, ‘competition’ between different individuals within a peer group may increase in the rate of compliance with diet and exercise plans.

Individuals additionally are allowed to communicate with other individuals within the peer group. Individuals are granted access to the nicknames and respective ranking and scores of other individuals within their peer group. True names or contact information can be withheld, in order to protect the privacy of all individuals concerned. Thus, in effect the individuals may remain anonymous, which results in lowered possible discomfort for the individual 12 and increased veracity of his/her inputted responses.

Health graphics are also used to depict the reports of the individual. The image of the individual's character may be shown to grow slim or fat depending on the individual's responses. Similarly, the image of an individual's character may be shown to be more muscular and smart appearing with a higher health score for the individual 12. Animated characters of individuals who are regularly exercising may be shown to grow increasingly active. Health graphics do not necessarily have to depict humanoid or living objects. Animated images of weight scales may be used to depict the above information.

Comparative scoring may additionally be implemented in graphics. Images of multiple characters suitably are shown as a single image on the display unit of the remote system 20 with the clothes of the characters labeled in order to reveal their identity. Similarly, multiple animated characters representing different individuals are shown simultaneously on the display unit of the remote system 20. Of course, individuals who do not wish to take part in the comparative scoring system are provided with reports that depict only their scores.

In other embodiments of the invention, the individual 12 inputs responses to queries on a PDA, and synchronizes the inputted information with that on the system at regular intervals of time. Using a PDA, the individual 12 advantageously does not need to communicate with server 30 each time he or she wishes to input data into system 10.

Finally, any person, such as the analyst 56 may be granted access to the system 10. The analyst 56 may be a researcher who wishes to test the efficacy of a new behavioral modification strategy or a new drug to achieve weight loss in individuals. Alternatively, the analyst 56 could be the representative of a company that manufactures exercise and workout equipment, or a company that markets health foods or drugs that help in weight reduction.

It will be clear to one skilled in the art that the above invention may be altered in many ways without departing from the scope of the invention. Various relative arrangements of inputs and processing means are possible for generating personalized health content. In addition to general health improvement, various diseases and behaviors are amenable to preventive care according to a method of the present invention, including asthma, hypertension, cardiovascular disease, eating disorders, HIV, mental health disorders, smoking, and drug or alcohol abuse.

While the preferred embodiment of the invention has been illustrated and described, as noted above, many changes can be made without departing from the spirit and scope of the invention. Accordingly, the scope of the invention is not limited by the disclosure of the preferred embodiment.

1. A system for processing general personal health information associated with at least one individual, the system comprising:
a plurality of remote devices, each remote device being associated with an individual;
at least one server to store content for communication to the remote devices, the content being selected from the group consisting of health information, non-health information, and entertainment content; and
a computer network operatively connected to the at least one server and the plurality of remote devices to facilitate communication of selected content to the individual via the associated remote device.

2. The system of claim 1, wherein data associated with each individual is collected, the collected data being selected from the group consisting of demography of the individual, personal health of the individual, familial health of the individual, and personalization factors associated with the individual.

3. The system of claim 2, wherein the system generates at least one individual profile selected from the group consisting of a health news profile associated with the individual, a preferred news profile associated with the individual, and an entertainment content profile associated with the individual.

4. The system of claim 3, wherein upon modification of the individual profile the system modifies health content provided to the individual and individual content monitored that is associated with the particular individual.

5. The system of claim 4, wherein the content comprises health education that is customized to the individual profile, and the relationship of health to many activities of daily living is explained to the individual.

6. The system of claim 4, wherein the content monitored comprises responses from the individual that are analyzed, scored and presented to the individual in a variety of reporting formats.

7. The system of claim 6, wherein in one reporting format, the individual's health status is followed in time and represented on a graph.

8. The system of claim 7, wherein the health status is color-coded and represented as such on a calendar.

9. The system of claim 7, wherein the individual user is represented pictographically as at least one of an image and an animated character, wherein when represented as an image, the image portrays health status and, when represented as an animated character, the actions portray the health status.

10. The system of claim 7, wherein the health status of an individual is ranked or graded after comparing it with other users similarly enrolled into the system.

11. The system of claim 1, wherein the server defines a personalized portal and the content accessed by the individual from the personalized portal is screened for related healthcare information by the use of Natural Language Processing algorithms.

12. A method for processing general personal health information associated with at least one individual, the method comprising:
providing a plurality of remote devices, each remote device being associated with an individual;
storing content on at least one server for communication to the remote devices, the content being selected from the group consisting of health information, non-health information, and entertainment content; and
communicating selected content to a remote device associated with the individual via a computer network operatively connected to the at least one server and the plurality of remote devices.

13. The method of claim 12, which comprises collecting data associated with each individual, the collected data being selected from the group consisting of demography of the individual, personal health of the individual, familial health of the individual, and personalization factors associated with the individual.

14. The method of claim 13, which comprises generating at least one individual profile selected from the group consisting of a health news profile associated with the individual, a preferred news profile associated with the individual, and an entertainment content profile associated with the individual.

15. The method of claim 14, wherein upon modification of the individual profile the method modifies health content provided to the individual and modifies content monitored that is associated with the particular individual.

16. The method of claim 15, wherein the content comprises health education that is customized to the individual profile, and the relationship of health to many activities of daily living is explained to the individual.

17. The method of claim 15, which comprises representing the individual user pictographically as at least one of an image and an animated character, wherein when represented as an image, the image portrays health status and, when represented as an animated character, the actions portray the health status.

18. A system for processing general personal health information associated with at least one individual, each individual being associated with a remote device, the system comprising:
means for storing content on at least one server for communication to the remote devices, the content being selected from the group consisting of health information, non-health information, and entertainment content; and
means for communicating selected content to the individual via an associated remote device via a computer network operatively connected to the at least one server and the plurality of remote devices.

19. A computer-readable medium for executing a sequence of instructions that, when executed by the computer, cause the computer to:
process general personal health information associated with at least one individual, each individual being associated with a remote device;
store content on at least one server for communication to the remote devices, the content being selected from the group consisting of health information, non-health information, and entertainment content; and
communicate selected content to the individual via an associated remote device via a computer network operatively connected to the at least one server and the plurality of remote devices.
